(19) United States
(12) Patent Application Publication
(10) Pub. No.: US 2022/0233987 A1
(43) Pub. Date: Jul. 28, 2022

(54) ULTRAVIOLET RADIATION MATRIX MEMBRANE APPARATUS, SYSTEM AND METHODS THEREOF

(71) Applicant: CAT 2020, INC., NAPLES, FL (US)

(72) Inventors: TIMOTHY M. GORMAN, NAPLES, FL (US); CATHERINE BLACK GORMAN, NAPLES, FL (US); CATHERINE GORMAN CORDOVA, ESTERO, FL (US); MITCHELL L. CORDOVA, ESTERO, FL (US)

(21) Appl. No.: 17/569,333

(22) Filed: Jan. 5, 2022

Related U.S. Application Data
(60) Provisional application No. 63/134,052, filed on Jan. 5, 2021.

(51) Int. Cl. B01D 46/00; B01D 46/54; A61L 9/20; B60H 3/06

(52) U.S. Cl. CPC B01D 46/0028; B01D 46/543; A61L 9/20; A61L 2209/14; B01D 2279/65; B01D 2279/40; B60H 3/0608

(57) ABSTRACT

The present disclosure is directed to a novel redesigned or retrofitted cabin air filtration system utilizing an ultraviolet light, radiation, or an energy matrix membrane and related technology to eliminate toxins such as viruses, bacteria, mold, fungi, rickettsia, protozoa, etc. from the air system in a vehicle, such as an automobile, marine vehicle airplane, etc., thereby improving the current cabin air filter functionality and providing a sterile air distribution environment throughout the cabin of the vehicle or small area.

### PRIORITY STATEMENT

The present application claims priority to U.S. Provisional Patent Application Ser. No. 63/134,052, filed Jan. 5, 2021, entitled “Ultraviolet Radiation Matrix Membrane Apparatus, System And Methods Thereof”, which is hereby incorporated by reference in its entirety.

### FIELD OF THE DISCLOSURE

The present disclosure relates to a cabin air filtration system that utilizes ultraviolet radiation to sanitize the air inside a vehicle or other small area, and in particular, the present disclosure relates to a novel redesigned or retrofitted cabin air filtration system utilizing an ultraviolet light, radiation or energy (sometimes referred to as blue light) matrix membrane and related technology to eliminate toxins such as viruses, bacteria, mold, fungi, rickettsia, protozoa, etc. from the air system, thereby improving the current cabin air filter functionality and providing a sterile air distribution environment throughout the cabin of a vehicle or small area.

The present disclosure relates to, inter alia, cabin air filtration systems, and in the preferred embodiment, the novel cabin air filtration systems comprises a flexible matrix membrane made up of a matrix and ultraviolet lights and/or Ultraviolet (ultraviolet or UV) radiation, which is incorporated into, by sewing or weaving, etc., or replaces completely standard or current cabin air filters for automobiles and other vehicles. The present disclosure incorporates ultraviolet light into a standard or existing cabin air filter, either by sewing or weaving the flexible matrix membrane into an existing cabin air filter or incorporating the flexible matrix membrane into a vehicle filter during the manufacturing process, thereby creating the novel cabin air filter.

The present disclosure relates to properly cleansing and sterilizing cabin air through the use of ultraviolet radiation. As detailed herein, UV radiation is a form of electromagnetic radiation with a wavelength from about 10 to 400 nanometers, and a corresponding frequency from around 30 PHz to 750 THz. The lower wavelength limit of human vision is approximately 400 nm, so ultraviolet rays are invisible to most humans. In particular, UV light at C, B and A wavelengths has been shown to disinfect and decontaminate surfaces, air, and water, and UV light at these three different wavelengths can sterilize and clean bacteria. Specifically, it is the UV-C region (100-280 nm) of the UV spectral range that is utilized in the preferred embodiment. With respect to disinfection, the optimum UV-C wavelength occurs in the region of 250 nm to 262 nm.

In use, the cabin air filter, utilizing the ultraviolet matrix membrane technology is installed into the air circulation system in the vehicle in the location of the standard system filter, thereby cleaning and sanitizing the air as it passes through the ultraviolet light matrix membrane technology filter. The original function of the filter material, to remove particulates from the air, will not be compromised and will continue to work in conjunction with the novel cabin air filter.

Notwithstanding the preferred embodiment above, the present disclosure relates to other types of motor vehicles and small areas that utilize air recirculation systems that use, can incorporate or can be optimize by the use of a standard air filter. Incorporating the present disclosure into these vehicles and/or small areas can eliminate toxins from the air, thereby cleaning and sterilizing the air being recirculating throughout the vehicle or area system. Toxins being cleansed or sterilized using the novel ultraviolet light matrix membrane technology include but are not limited to viruses, bacteria, mold, fungi, rickettsia, protozoa, etc. This capability can be accomplished while the novel cabin air filter continues to remove pollutants, pollen, dust, etc. as currently performed in standard cabin air filtration systems.

### BACKGROUND OF THE DISCLOSURE

In the cabin air filtration industry there is a need for a device or a system that will sufficiently cleanse and sanitize the air inside a cabin, vehicle or other small area, especially given the COVID-19 environment. The device or system must perform and accomplish this objective while continuing to remove particulate matter such as pollutants, pollen, and dust from the air inside that same area. In particular, there is a need for the device or system to accomplish these goals without requiring intrusive redesigns or retrofits to the existing cabin air filter system. The device or system must efficiently and effectively eliminate toxins such as viruses, bacteria, molds, fungi, rickettsia, protozoa, etc. from the air system to improve and/or sterilize the air being distributed throughout the cabin of the vehicle (for example, automotive or marine) or small area.

An air purifier or air cleaner is a device that removes particulates or contaminants from the air in a room with the goal of improving the air quality of that room. Air cleaners or air filtration systems usually use filters to accomplish these goals and are well known to be beneficial to those who have allergies or have medical conditions, such as asthma, etc. The filter is placed or located in the system such that the airflow will draw the pollutant air through the filter to capture these airborne particulate matter. The filter will then block the particulate matter and allow the remaining air to travel through the filter, thereby reducing or eliminating particulate matter such as pollutants, pollen, dust, tobacco smoke, etc. from the air. The filter is able to block certain particulates based on their size and different filters will block different sized pollutants.

The particulate air filter can be composed of fibrous or porous materials which act to remove the solid particulates. Additionally, filters containing a catalyst such as charcoal or carbon may also remove odors and pollutants. Other materials can be used in filters, such as foam, pleated paper, or spun fiberglass, and in some cases fibers are statically charged to attract dust particles. Also, high efficiency particulate arrester filters, or HEPA filters are a type of air filter that, due to high standards, are used in clean rooms for integrated circuit fabrication, medical facilities, automobiles, aircraft and homes. Whereas course filters can block particulate matter about 5.0 microns or larger, and fine filters can block particulate matter as small as 1.0 microns, HEPA filters can eliminate particulate matter as small as 0.3 microns.

As mentioned, some of these different filters and systems are utilized in vehicles, such as automobiles or marine vehicles, for reducing or eliminating pollutants from the cabin air. These cabin air filters are made from paper and/or carbon fiber and are placed in the vehicle's air distribution system (heating and air conditioning system) to reduce or eliminate the particulate matter. While most people understand the engine filter system, since the vehicle's air distribution system is used in conjunction with the heating and air conditioning functionality, in most cases the vehicle occupants do not even know that cabin air is being filtered. Usually, the cabin air filter is located behind or near the glove box or glove compartment, and over time, just like any other filtering system, the cabin air filter will break down and/or reduce its functionality and need to be replaced. Nonetheless, vehicle owners and the individuals servicing the vehicles often fail to replace the air filters.

However, as detailed herein, the cabin air filter merely removes particulate matter of a certain size from the cabin air. The cabin air filter does not cleanse or sterilize the cabin air, which is necessary to remove viruses, bacteria, molds, etc. from the cabin air. Some cabin air filters attempt to provide multiple functions by layering the cabin air filter. For example, a filter may eliminate dust and pollen with a particulate filtration layer, trap mold and harmful gases with activated charcoal, absorb bad smells with baking soda, and even weaken germs, bacteria, and viruses with a different particulate filtration layer. However, this multilayered process still does not kill viruses, bacteria and germs.

Ultraviolet radiation was discovered in 1801, and the sterilizing effect of short-wavelength radiation by killing bacteria was discovered in 1878. By 1903, the most effective wavelengths were known to be around 250 nm, and the effect of ultraviolet radiation on DNA was established in 1960.

Ultraviolet radiation is shorter than that of visible light, but longer than X-rays; and ultraviolet radiation is present in sunlight, although the chemical and biological effects of ultraviolet energy is greater than just the heating effect. For humans, suntan and sunburn are familiar effects of exposure of the skin to ultraviolet energy, along with an increased risk of skin cancer.

In particular, Ultraviolet radiation disinfection has been used for decades and has various benefits including low environmental impacts, and small space requirements. Ultraviolet germicidal irradiation (UVGI) is a well-established mechanism for disinfecting against various viruses, bacteria, fungi, etc. that cause acute respiratory syndromes thereby helping decrease the spread of infectious diseases.

Along those lines, Ultraviolet light at C, B and A wavelengths has been shown to disinfect and decontaminate surfaces, air, and water, and at these three different wavelengths, UV light can sterilize and clean bacteria. Specifically, it is the UV-C region (100-280 nm) of the UV spectral range that is utilized, and with respect to disinfection, the optimum UV-C wavelength occurs in the region of 250 nm to 262 nm.

As wavelengths increase, germicidal efficacy begins to decay exponentially. The mechanism by which UV-C radiation disinfects is by inactivating the target microbe by damaging their DNA structure. Because of these properties, it is not surprising that UVGI has been widely used to disinfect air, water, and various surfaces across many industries (e.g. food, healthcare, water purification). Traditionally, disinfection has been delivered using low-pressure mercury discharge lamps where shortwave UV-C radiation (100-258 nm wavelengths) is emitted primarily at 254 nm to inactivate microorganisms.

Additionally, with the on-going COVID-19 pandemic for the past two years it has been recently discovered that UV-C light, blue light that radiates at shorter wavelengths with higher frequencies, can disinfect air, various surfaces, and water by against the SARS CoV2 virus by breaking down its outer protein coating. It has been also recently confirmed that blue light, between 380-500 nm, has germicidal qualities when issued at wavelength range of range of 400 to 450 nm.

With significant advances made in light emitting diode (LED) technology, UV-C radiation can be delivered much more efficiently and safely compared to traditional mercury lamps. Specifically, UV-C LEDs contain no hazardous material, can be switched on or off instantaneously without cycling limitations, have lower heat consumption, and are more durable, among other benefits. UV-C exposure through diode technology removes microorganism when exposed to the light at milijoule (mJ) per square centimeter of fluence (UV dosage). For example, tests have shown that Staphylococcus aureus exposed to 52 mJ for less than 5 seconds is inactivated. Other research has demonstrated UV diodes were effective in sterilizing pathogens including COVID-19.

Similarly, studies have shown that UV-C light exposure disinfects Methicillin-resistant Staphylococcus aureus (MRSA) at 254-nm UV-C dose of 22,000 μWs/cm2 for 45 min. Because UV-C lighting can degrade certain materials such as plastic, certain textiles, and polymers, the FDA claims that UV LEDs have peak wavelengths at 265 nm, 273 nm, and 280 nm. However, other data has shown that far-UV-C wavelengths at 222 nm inactivates the Coronavirus. It has been demonstrated that low doses of 1.7 and 1.2 mJ/cm2 inactivated 99.9% of aerosolized Coronavirus 229E and OC43, respectively.

As all human coronaviruses have similar genomic sizes, far-UV-C light would be expected to show similar inactivation efficiency against other human coronaviruses including SARS-CoV-2. Based on the beta-HCoV-OC43 results, continuous far-UVC exposure in occupied public locations at the current regulatory exposure limit (˜3 mJ/cm2/hour) would result in ˜90% viral inactivation in ˜8 minutes, 95% in ˜11 minutes, 99% in ˜16 minutes, and 99.9% inactivation in ˜25 minutes. Thus, it is further suggested that while staying within current regulatory dose limits, low-dose-rate far-UVC exposure can potentially safely provide a major reduction in the ambient level of airborne coronaviruses in occupied public locations.

Despite its positive disinfecting properties, UV light exposure can damage human skin or eyes. In addition, the porousness and material of a surface for disinfection can influence the UV lighting. It has been shown that that certain materials, such as polypropylene, has varying results in disinfection from UV-C radiation based on its density. It has been demonstrated that layers made from polypropylene (PP), hydrophobic in nature, resulted in higher disinfection efficiency than layers composed of polyethylene terephthalate (PET-P), hygroscopic in nature. Many cabin air filters are made from polyester material, which may require longer dosage and exposure due to density.

As described herein, many practical applications of ultraviolet radiation derive from its interactions with organic molecules. For example, short-wave ultraviolet energy damages DNA and sterilizes surfaces with which it comes into contact. In particular, short-wave ultraviolet energy is known to disrupt DNA base pairing, which causes formation of pyrimidine dimers. These pyrimidine dimers, if not corrected, can lead to melanoma in humans, but also lead to the inactivation of bacteria, viruses, and protozoa. As such, when used properly, ultraviolet radiation can be an effective method for eliminating or inactivating bacteria and viruses.

Accordingly, ultraviolet radiation can be used in germicidal irradiation in air purification systems. For example, UVGI systems can be used to sterilize the air that passes through the system and by UV lamps through a forced air system. UVGI systems used for air purification can be freestanding units with shielded ultraviolet lamps that use a fan to force air past the ultraviolet light. Other systems are installed in forced air systems so that the circulation for the premises moves micro-organisms past the lamps. The key to this form of sterilization is the placement of the ultraviolet lamps and a good filtration system to remove the dead micro-organisms.

These forced air systems may however, create areas in the system that will be shaded from the ultraviolet light. In certain instances, placing the ultraviolet lamp at or near the coils and drain pan of the cooling system may keep viruses, etc. from forming in these naturally damp places. The most effective method for treating the air rather than the coils is in-line duct systems, these systems are placed in the center of the duct and parallel to the airflow.

Attempts have been made to utilize ultraviolet radiation with a filter, as described in U.S. Pat. No. 5,837,207, to Summers, entitled Portable Germicidal Air Filter, and which is incorporated herein by reference. The '207 patent discloses a lightweight portable germicidal air filter for home and personal use. The air filter includes a cabinet which houses an electrostatic air filter, an ultraviolet lamp and a parabolic reflector or a convex lens for focusing the ultraviolet radiation emitted by the lamp on an upstream side of the air filter. The reflector or the lens is constantly oscillated to systematically sweep the upstream side of the filter with germicidal levels of radiation. A fan located adjacent the downstream side of the filter draws air through the filter and impels it out through areas for air outlet in the sidewalls of the cabinet. According to the inventor, the advantage is a simple, lightweight germicidal air filter with few moving parts which is inexpensive to manufacture.

Another attempt to effectively irradiate viruses and bacteria is described in U.S. Pat. No. 6,783,578, to Tillman, entitled Air Purification Unit, and which is incorporated herein by reference. The '578 patent discloses an air purification apparatus that includes a HEPA or ULPA cartridge filter and an ultraviolet lamp mounted axially, therein extending from a closed end of the cartridge. An air channeling housing is provided for removably mounting the combination filter and lamp assembly, for ingress of an air stream through the open end of the combination filter and lamp assembly and for egress of the air stream through the filter medium. A fan can be mounted in the housing either upstream or downstream of the combination filter to provide the means for flowing air through the housing for decontamination by both the filter and ultraviolet irradiation provided by the combination filter and lamp assembly. With this arrangement, all contaminants trapped on the filter are within the combination filter and lamp assembly for constant illumination by the ultraviolet lamp to destroy bacteria both in the incoming air stream and while resident on the interior of the filter assembly. As disclosed, when the apparatus is installed in an air stream, the air moves into the tubular filter where contaminants are killed by the ultraviolet lamp either before or after capture on the interior of the tubular filter before egress through the filter medium.

Yet another attempt to effectively irradiate viruses and bacteria is described in U.S. Pat. No. 9,696,049, to Metteer, entitled Modular Ductwork Decontamination Assembly, and which is incorporated herein by reference. The '049 patent discloses a modular ductwork assembly that decontaminates an air stream circulating within a heating, ventilation and air conditioning (HVAC) system. The assembly includes one or more of (a) an ionizing module for removing particulates from the air stream, (b) a sterilization module for neutralizing airborne pathogens present in the air stream, (c) an ozone treatment module for neutralizing pathogens or odoriferous or gaseous constituents or volatile organic compounds (VOCs) present in the air stream, optionally (d) baffles for slowing and disrupting the flow rate and promoting turbulence in the air stream traveling through the modules, optionally (e) a fan module for directing a treated air stream, optionally (f) an ozone sensor, optionally (g) a monitoring or ozone control means, and optionally (h) a means of delivering and repurposing generated ozone. Each of the modules is arranged substantially adjacent to at least one of the other modules.

Another attempt to effectively irradiate viruses and bacteria is described in U.S. Patent Application No. 20050142047, to Baik et al., entitled Hybrid-Type Air Purifier For An Automobile, and which is incorporated herein by reference. The '047 patent application discloses air purifier systems particularly suitable for motor vehicles that comprise an air flow passage; a glow discharging device; a photo-catalyst device; and a negative ion generator. Preferred air purifiers comprise a glow discharging device having mesh-structured electrodes which can purify air via dielectric barrier discharge, a photo-catalyst device that can further purify the air firstly purified by the glow discharging device, and a negative ion generator having nano tubes, which can treat the purified air with negative ions.

All of the references described herein pertain to utilizing ultraviolet radiation to eliminate toxins from air circulation systems. However, none of the systems disclosed provide the features, functionality, simplicity, ease of use and effectiveness of the present disclosure. In particular, none of the existing patents pertaining to purifying or sanitizing air use UV-C LEDs. The advantage here is that low voltage, high efficiency light emitting diodes can be used to purify air through the car cabin when the vehicle is running. Another advantage is that the UV-C wavelengths are 250-262 nm which is ideal for sanitation. Moreover, these wavelengths are not ozone depleting compared to older UV germicidal radiation lamp technology, thereby creating a safer and more efficient means to purify air through a cabin filter.

As such, there is no apparatus, system or method relating to a novel cabin air filter utilizing ultraviolet light matrix membrane technology that will sufficiently cleanse and sanitize the air inside a cabin, vehicle or other small area, while continuing to remove particulate matter from that same air. In particular, there is a need for the device or system to accomplish these goals without requiring intrusive redesigns or retrofits to the existing cabin air filter system. The device or system must efficiently and simply eliminate toxins such as viruses, bacteria and molds from the air system to improve and/or sterilize the air being distributed throughout the cabin of the vehicle or small area. The present disclosure satisfies these needs.

### SUMMARY OF THE DISCLOSURE

In order to solve the above-mentioned shortcomings in cabin air filter design, the present disclosure pertains to a novel redesigned or retrofitted cabin air filtration system that incorporates or utilizes flexible ultraviolet light matrix membrane technology to eliminate toxins such as viruses, bacteria and molds from the air system. This disclosure pertains to improving the standard cabin air filter functionality while also providing a sterile air distribution environment throughout the cabin of a vehicle or small area.

An objective of the present disclosure is to provide a novel and improved cabin air filtration system which can be incorporated into or replace completely standard cabin air filters for automobiles, marine vehicles, and other vehicles, and other areas in which air is forced through a standard filter. The present disclosure incorporates ultraviolet light or energy into a standard or existing cabin air filter, either by sewing, weaving or otherwise incorporating a flexible ultraviolet radiation matrix membrane into the existing cabin air filter or incorporating the novel ultraviolet radiation into a vehicle filter during a manufacturing process.

Another objective of the present disclosure is to provide a novel and improved cabin air filtration system which can be incorporated into or replace completely standard cabin air filters for automobiles, marine vehicles, and other vehicles and other areas in which air is forced through a standard filter. The present disclosure incorporates ultraviolet light or energy, including Ultraviolet light at C, B and A wavelengths, into a standard or existing cabin air filter, either by sewing, weaving or otherwise incorporating a flexible ultraviolet radiation matrix membrane in the specific wavelength into the existing cabin air filter or incorporating the novel ultraviolet radiation into a vehicle filter during a manufacturing process.

Another objective of the present disclosure pertains to the installation of the cabin air filter utilizing the flexible ultraviolet light matrix membrane technology into the air circulation system in the vehicle or other area in the location of, or replacing completely, the standard system filter, thereby cleaning and sanitizing the air as it passes through the novel cabin air filter.

Yet another objective of the present disclosure pertains to allowing the original function of the filter material in the standard filter, to continue to remove particulates from the air, without being compromised due to the present disclosure, and as such will continue to work in conjunction with the novel cabin air filter.

Another objective of the present disclosure is that by incorporating the present disclosure into a vehicle, such as an automobile or marine vehicle, and/or small area, it will optimally and effectively eliminate toxins from the air by cleaning and sterilizing the air being recirculating throughout the vehicle or area. The toxins being cleansed or sterilized using the novel cabin air filter technology include but are not limited to viruses, bacteria, mold, etc. and this capability can be accomplished while continuing to remove pollutants, pollen, dust, etc. as currently being performed in standard filtration systems.

As such, the present disclosure solves the problems facing the automobile, marine and other industries as described herein. The present disclosure incorporates a solution for economically and efficiently removing both toxins and particulate matter from the inside of a vehicle and/or small area.

These and other aspects, features, and advantages of the present disclosure will become more readily apparent from the attached drawings, the detailed description of the preferred embodiments, and the recited claims, which follow.

### DRAWINGS

The preferred embodiments of the disclosure will be described in conjunction with the appended drawings provided to show and not to the limit the disclosure, where like designations denote like elements, and in which:

FIG. 1A shows a prior art cabin air filter for removing particulate matter from the cabin of a vehicle.

FIG. 1B shows a novel cabin air filter for removing particulate matter from the cabin of a vehicle in accordance with the present disclosure.

FIG. 2 shows an ultraviolet radiation matrix membrane for a cabin air filter in accordance with the present disclosure.

FIG. 3 shows a cabin air filter incorporating ultraviolet radiation matrix membrane technology in accordance with the present disclosure.

FIG. 4 shows a cabin air filter incorporating ultraviolet radiation matrix membrane technology in accordance with the present disclosure.

FIG. 5 shows a cabin air filter incorporating ultraviolet radiation matrix membrane technology in accordance with the present disclosure.

FIG. 6 shows a non-carbon fiber cabin air filter incorporating ultraviolet radiation matrix membrane technology in accordance with the present disclosure.

FIG. 7 shows a non-carbon fiber cabin air filter incorporating ultraviolet radiation matrix membrane technology in accordance with the present disclosure.

FIG. 8 shows a non-carbon fiber cabin air filter incorporating ultraviolet radiation matrix membrane technology in accordance with the present disclosure.

FIG. 9 shows a non-carbon fiber cabin air filter incorporating ultraviolet radiation matrix membrane technology in accordance with the present disclosure.

FIG. 10 shows a carbon fiber cabin air filter incorporating ultraviolet radiation matrix membrane technology in accordance with the present disclosure.

FIG. 11 shows a carbon fiber cabin air filter incorporating ultraviolet radiation matrix membrane technology in accordance with the present disclosure.

FIG. 12 shows a carbon fiber cabin air filter incorporating ultraviolet radiation matrix membrane technology in accordance with the present disclosure.

FIG. 13 shows a close up view of a cabin air filter incorporating ultraviolet radiation matrix membrane technology in accordance with the present disclosure.

FIG. 14 shows a close up view of a cabin air filter incorporating ultraviolet radiation matrix membrane technology in accordance with the present disclosure.

FIG. 15 shows a close up view of a cabin air filter incorporating ultraviolet radiation matrix membrane technology in accordance with the present disclosure.

FIG. 16 shows a close up view of a cabin air filter incorporating ultraviolet radiation matrix membrane technology in accordance with the present disclosure.

FIG. 17 shows a close up view of a cabin air filter incorporating ultraviolet radiation matrix membrane technology in accordance with the present disclosure.

FIG. 18 shows a close up view of a cabin air filter incorporating ultraviolet radiation matrix membrane technology in accordance with the present disclosure.

FIG. 19 shows an extreme close up view of a cabin air filter incorporating ultraviolet radiation matrix membrane technology in accordance with the present disclosure.

FIG. 20 shows a carbon fiber cabin air filter incorporating ultraviolet radiation matrix membrane technology in accordance with the present disclosure.

FIG. 21 shows a carbon fiber cabin air filter incorporating ultraviolet radiation matrix membrane technology in accordance with the present disclosure.

FIG. 22 shows a carbon fiber cabin air filter incorporating ultraviolet radiation matrix membrane technology in accordance with the present disclosure.

FIG. 23 shows a carbon fiber cabin air filter incorporating ultraviolet radiation matrix membrane technology in accordance with the present disclosure.

FIG. 24 shows a carbon fiber cabin air filter incorporating ultraviolet radiation matrix membrane technology in accordance with the present disclosure.

### DETAILED DESCRIPTION

One way to properly cleanse and sterilize cabin air is through the use of ultraviolet radiation. Ultraviolet (UV) radiation is a form of electromagnetic radiation with a wavelength from about 10 to 400 nanometers, and a corresponding frequency from around 30 PHz to 750 THz. The lower wavelength limit of human vision is approximately 400 nm, so ultraviolet rays are invisible to most humans.

Along those lines, Ultraviolet light at C, B and A wavelengths has been shown to disinfect and decontaminate surfaces, air, and water. UV light at these three different wavelengths can sterilize and clean bacteria. Specifically, the preferred embodiment utilizes the UV-C region (100-280 nm) of the UV spectral range. With respect to disinfection, the optimum UV-C wavelength occurs in the region of 250 nm to 262 nm.

As described herein, the present disclosure relates to a novel cabin air filter 10 and filtration system 100 utilizing a matrix membrane 12 that incorporates a matrix 14 with one or more ultraviolet lights 16 that generate ultraviolet radiation or energy, and the related technology. The matrix may also be flexible.

The technology is used to eliminate toxins such as viruses, bacteria, mold, fungi, rickettsia, protozoa, etc. from an air ventilation or circulation system 110 in a vehicle 18 (or other area), thereby improving the functionality of a standard cabin air filter 20 and providing a sterile air distribution environment throughout the cabin area 22 of the vehicle 18 or other small areas (not shown). The novel cabin air filter 10 can be created by redesigning the standard cabin air filter 20 and manufacturing process altogether, or by retrofitting standard cabin air filters 20 to produce the novel cabin air filter 10 disclosed herein.

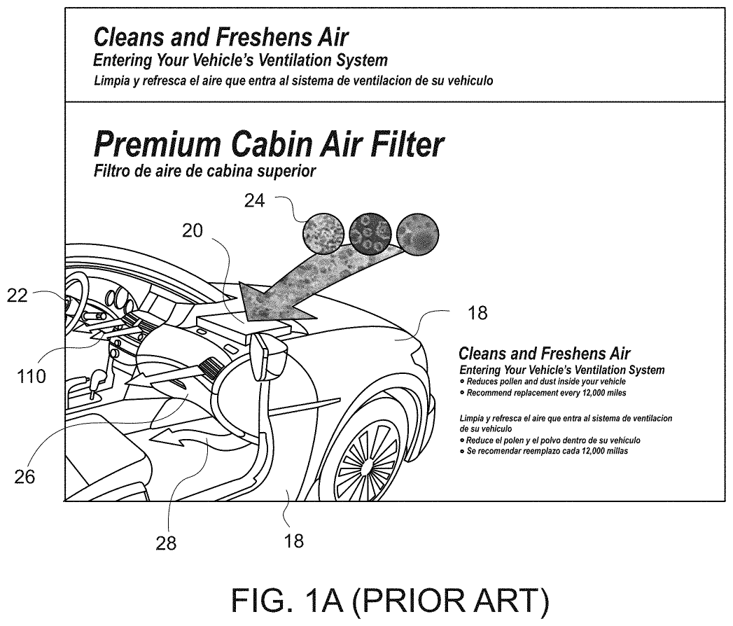

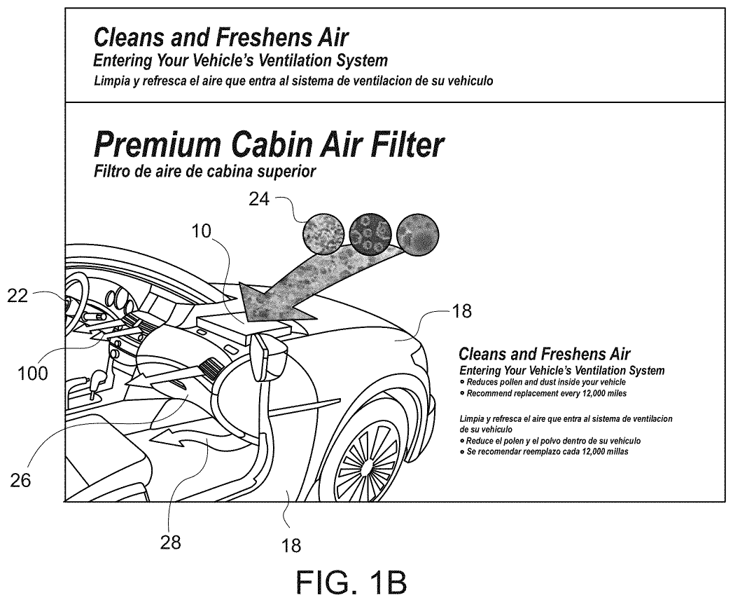

FIG. 1A shows a vehicle 18 and the cabin area 22 of that vehicle where passengers (not shown) usually sit. In the cabin area 22, air is circulated throughout the area 22 using a ventilation system 110, as understood by one having ordinary skill in the art.

As the air in the cabin area 22 is drawn into the ventilation system 110, the air circulates around the system 110 and ultimately passes through the standard cabin air filter 20, where particulate matter or pollutants 24, such as pollen, dust, etc. are caught by the standard cabin air filter 20 and thus removed from the air. As the air is further circulated through the ventilation system 110 and returned to the cabin area 22 of the vehicle 18, the passengers are not subjected to these pollutants 24 and can breathe fresh air.

The standard cabin air filter 20 is usually located or placed behind the glove box or glove compartment 26 so that replacement of the standard cabin air filter 20 is not difficult to perform. Also, based on this convenient location, the circulating air (see arrows) 28 can be easily accessed in the ventilation system 110 before being sent into the cabin area 22.

In the present disclosure, FIG. 1B shows a similar system 100 to the prior art system 110 in FIG. 1A. In FIG. 1B, the novel cabin air filter 10 is incorporated into a novel filtration systems 100 and replaces the standard cabin air filter 10 completely, or is used in conjunction therewith. As described herein, the present disclosure incorporates ultraviolet light or radiation 16 into a standard or existing cabin air filter 20 to create the novel cabin air filter 10. In the preferred embodiment, the UV radiation is in the UV-C region (100-280 nm) of the UV spectral range, and with respect to disinfection, the optimum UV-C wavelength occurs in the region of 250 nm to 262 nm.

The disclosed novel cabin air filter or matrix 10 can be manufactured to size, to fit in any pre-existing cabin air filter box, such as those found in most vehicles, including, but not limited to, automobile, truck, marine or airplane vehicles.

In the preferred embodiment, a UV-C LED matrix 10 would receive its power from the vehicle through for example, a USB plug or suitable power supply 32 depending on the application (e.g. automotive or marine, etc.), allowing the UV-C LED matrix 10 to line the cabin air filter box 26 and sanitize the air 28 before it passes through a standard cabin air filter 20. The novel cabin air filter or matrix 10 can also be battery powered (rechargeable or disposable).

As explained above, despite the positive disinfecting properties, UV light exposure can damage human skin or eyes. In addition, the porousness and material of a surface for disinfection can influence the UV lighting. It has been shown that that certain materials, such as polypropylene, has varying results in disinfection from UV-C radiation based on its density. It has been demonstrated that layers made from polypropylene (PP), hydrophobic in nature, resulted in higher disinfection efficiency than layers composed of polyethylene terephthalate (PET-P), hygroscopic in nature. Many cabin air filters are made from polyester material, which may require longer dosage and exposure due to density.

For this reason, the present disclosure teaches placement of the continuously powered low-voltage UV-C LED blue light matrix within the cabin air filter system within the automobile compartment places the device out of reach and allows for safe and protected air sanitation when the vehicle is running.

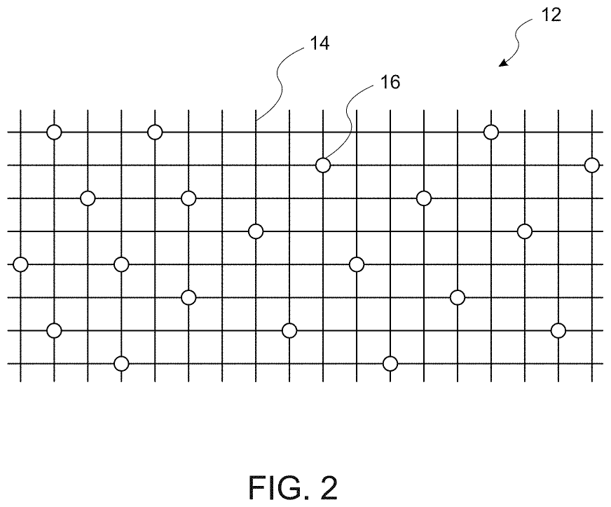

FIG. 2 shows the flexible matrix membrane 12 that is created by attaching or otherwise incorporating the ultraviolet lights 16 into a flexible matrix 14 from the power source 32. Although the preferred embodiment discloses a flexible matrix membrane 12 and matrix 14, both the matrix membrane 12 and the matrix 14 can be rigid depending on the particular cabin air filter design. Additionally, the flexible matrix membrane 12 can be a single strand incorporating the UV lights 16, not the two-dimensional version shown in FIG. 2, and separately, the matrix 14 may be incorporated into the cabin air filter without a separate matrix membrane 12 (such as a string of UV lights 16).

The flexible matrix membrane 12 (or the matrix 14) may also comprise a center portion to facilitate the attachment of the one or more ultraviolet lights 16 therein. The flexible matrix membrane 12 is then incorporated into the standard cabin air filter 10, either by sewing or weaving the flexible matrix membrane 12 (or matrix 14 or a string of UV lights 16) into or throughout a standard cabin air filter 20, or otherwise attaching the matrix membrane to create the novel cabin air filter 10. Additionally, the novel cabin air filter 10 can be designed and manufactured as a unit without the need to upgrade or retrofit a standard cabin air filter 20. Further, for certain applications, the matrix membrane 12 can be manufactured to be nonflexible.

In the preferred embodiment, the UV-C LED matrix 14, for example, can be woven within the pleats of the standard filter 20 to manufacture the novel air filter 10. This particular manufacturing process will allow for the air 28 that passes through the filter 10 to be sanitized and/or disinfected.

Once the matrix membrane 12 is attached to or incorporated into the standard cabin air filter 20 to create the novel cabin air filter 10, the novel cabin air filter 10 can be placed or located behind the glove box 26 or into the proper location for that particular vehicle 18. Once properly installed, the ventilation system 110 will force cabin air from the cabin area 22 through the filtration system 100 and through the novel cabin air filter 10. The novel cabin air filter 10 will then remove particulate matter 24 (utilizing the materials from the standard cabin air filter 20) and utilize the ultraviolet lights 16 on the matrix membrane 12 to clean and sterilize toxins 30, such as viruses, bacteria, mold, etc., as they passes through the novel cabin air filter 10.

The present disclosure relates to other types of motor vehicles and other areas that utilize circulation or ventilation systems 110 and that can incorporate a similar filtration system 100. Incorporating the present disclosure into these vehicles 18 and areas (not shown) can eliminate toxins 30 from the circulating air 28, thereby cleaning and sterilizing the air 28 being recirculating throughout the vehicle 18 or area system. This functionality and capability of the novel cabin air filter 10 can be accomplished while continuing to remove particulate matter, pollutants, pollen, dust, etc. 24 as currently being performed in standard filtration systems.

There are numerous different types of standard cabin air filters 20 that the present disclosure may be incorporated into to create the novel cabin air filter 19. Of course, the novel cabin air filter 10 can also be manufactured separately (as an OEM product), without the need to upgrade a standard cabin air filter 20. Additionally, the matrix membrane 12, or the string of UV lights 16, can be manufactured and sold separately as an aftermarket device for attaching or using in conjunction with to a standard cabin air filter 20.

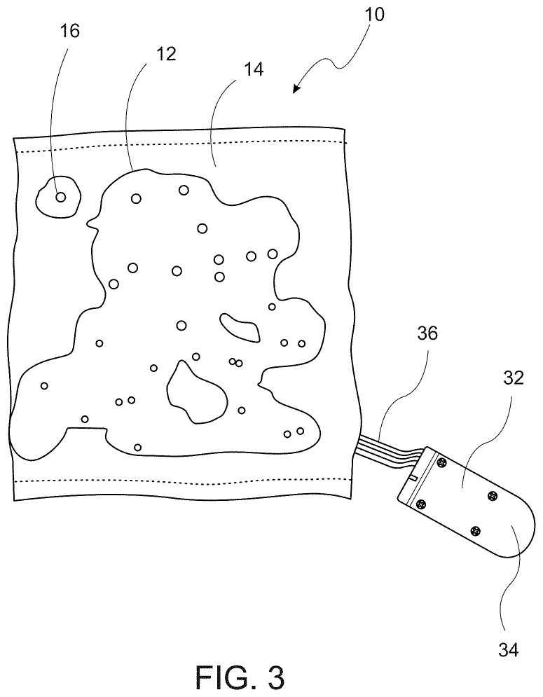

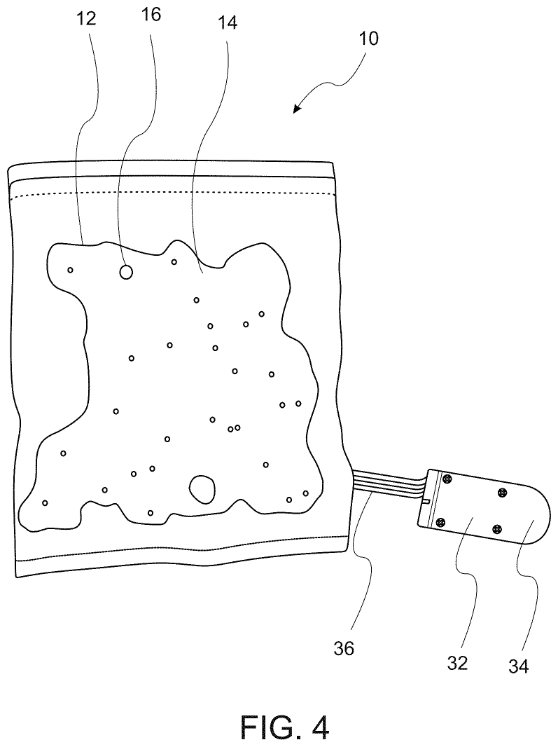

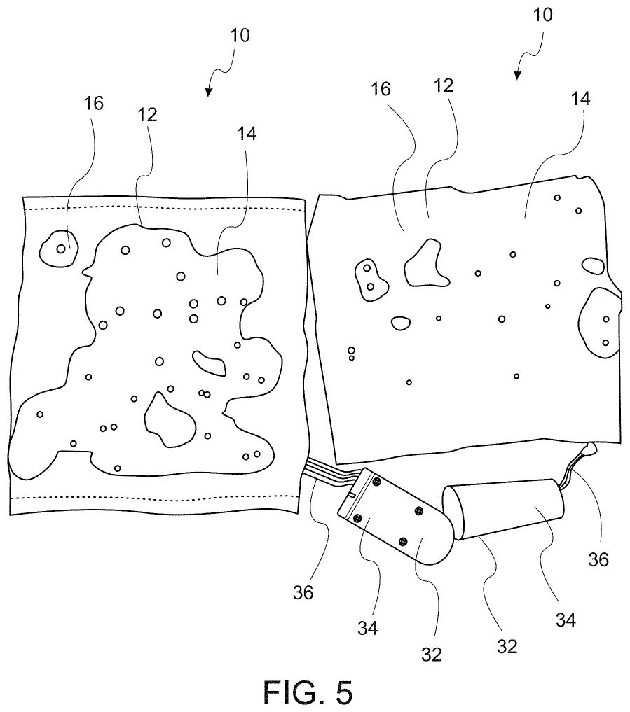

FIGS. 3 through 5 show a novel cabin air filter 10 incorporating the matrix membrane 12 and displaying the ultraviolet radiation or lights 16 attached to the matrix 14. The circulating air 28 will be forced across the novel cabin air filter thereby cleaning and sterilizing the circulating air 28.

Also illustrated are the power sources 32, such as a battery (not shown) and battery holder 34, along with the electrical connection 36 that is used to power the ultraviolet lights 16. In use in a vehicle 18, the electrical connection 36 can be incorporated into the power from the vehicle 18, such as the cigarette lighter (not shown) or other power connectors as understood by one having ordinary skill in the art.

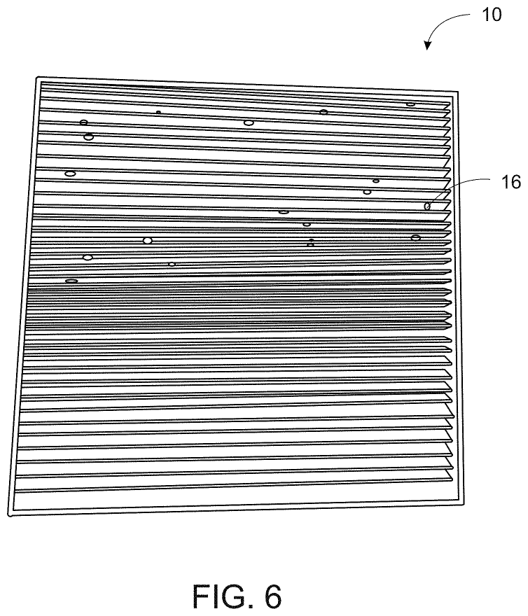

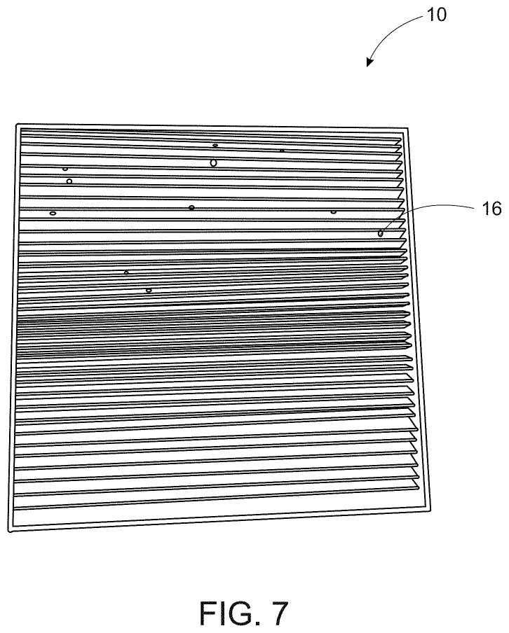

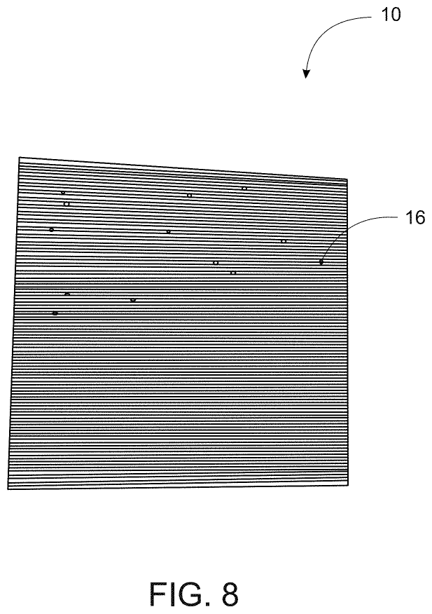

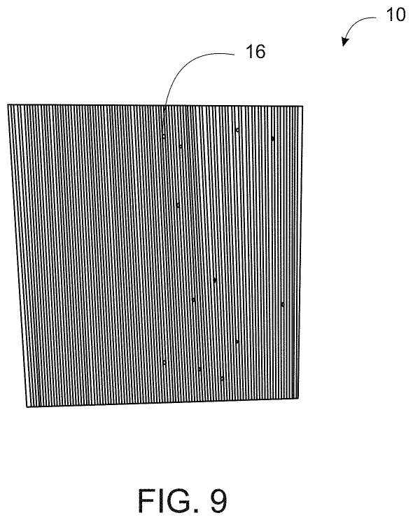

Additional types of novel cabin air filters 10, having non-carbon fiber filters, are illustrated in FIGS. 6 through 9 incorporating the matrix membrane 12 and displaying the ultraviolet radiation 16.

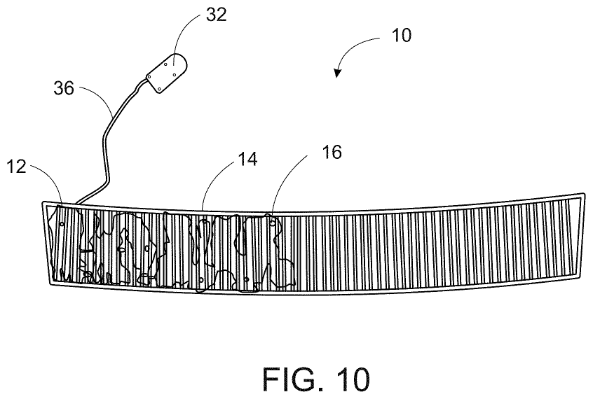

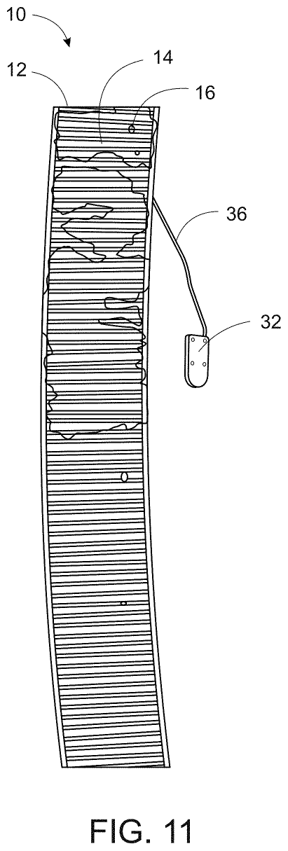

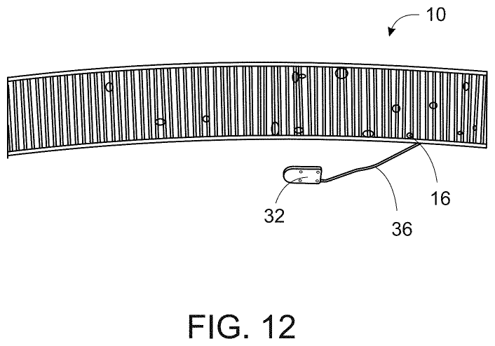

FIGS. 10 through 12 show novel cabin air filters 10, using carbon fiber, and displaying ultraviolet radiation 16 incorporating therein.

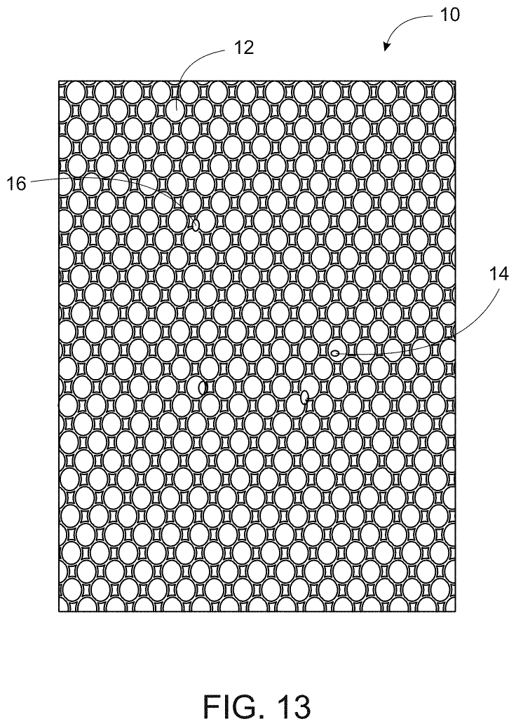

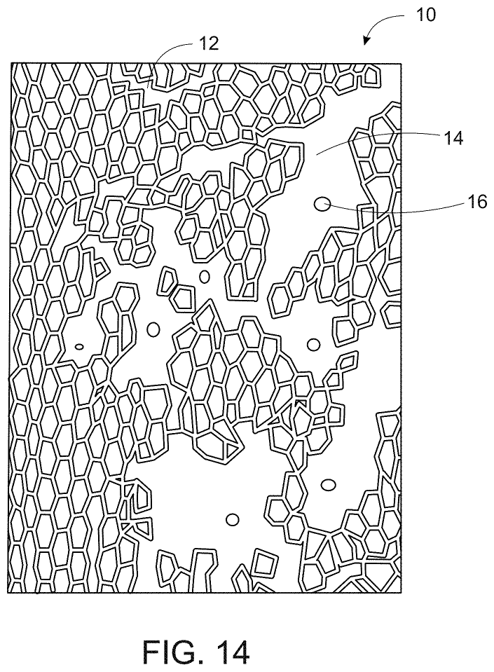

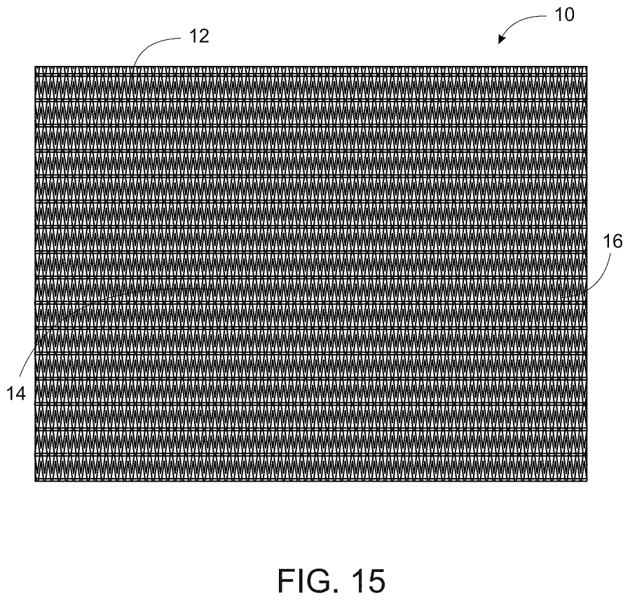

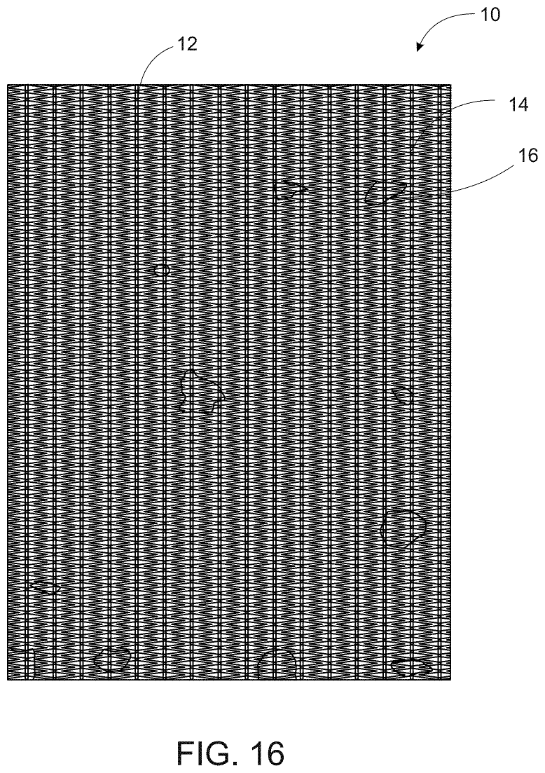

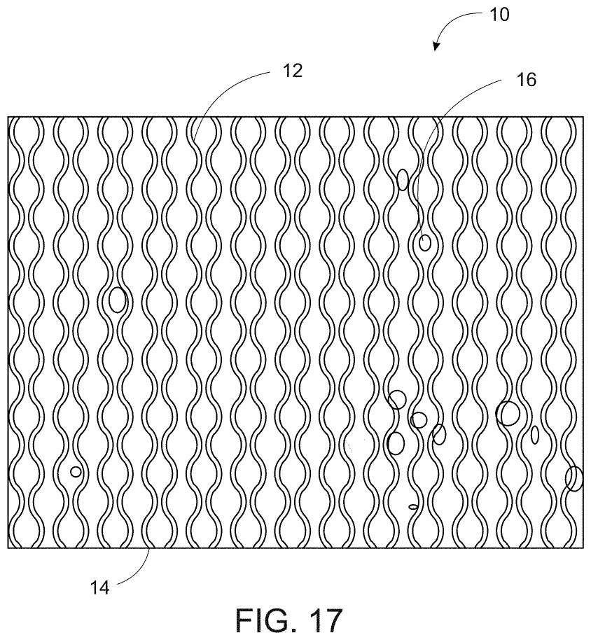

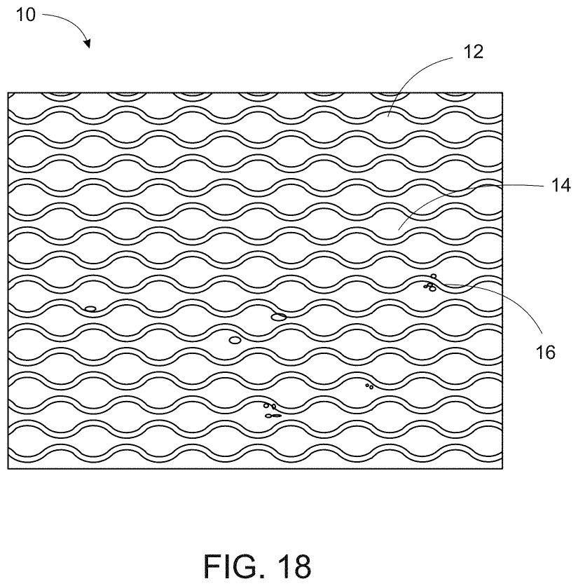

FIGS. 13 through 18 show novel cabin air filters 10 using various different materials for the matrix membrane 12. Each of these different matrix membranes 12 provide different types of openings or holes thereby providing varying levels of translucence or opaqueness. In all of these matrix membranes the matrix 14 (or matrices 14) and the ultraviolet lights 16 are captured by the matrix membrane 12, but allow for the ultraviolet lights or ultraviolet energy 16 to sanitize the air passing through the matrix membrane 12 of the novel cabin air filter 10.

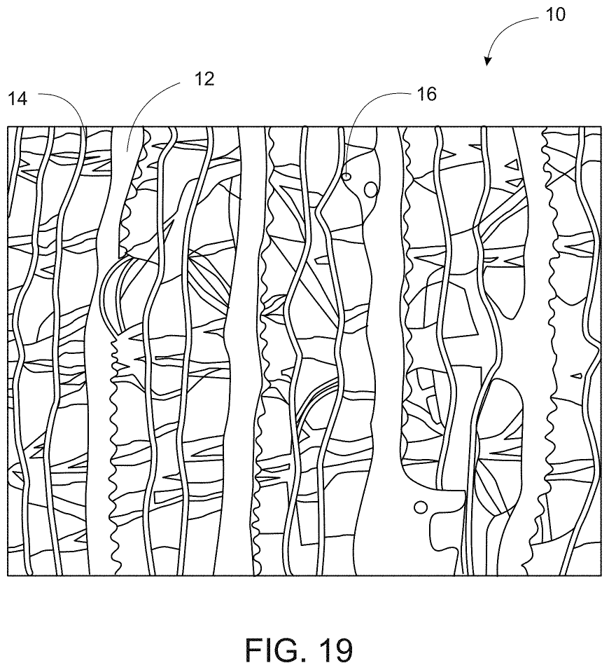

FIG. 19 is an extreme close up of the matrix membrane 12 of the novel cabin air filter 10 and more clearly displays the matrix 14 and the ultraviolet light and radiation 16 incorporating therein. As described herein, the matrix incorporates a flexible material facilitating the placement of the ultraviolet lights 16 and the flexibility of the matrix 14 to be used in many different combinations and embodiments.

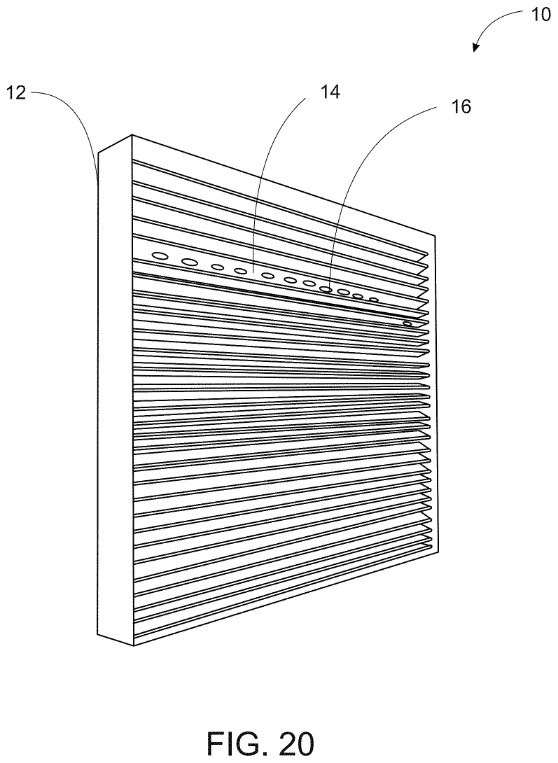

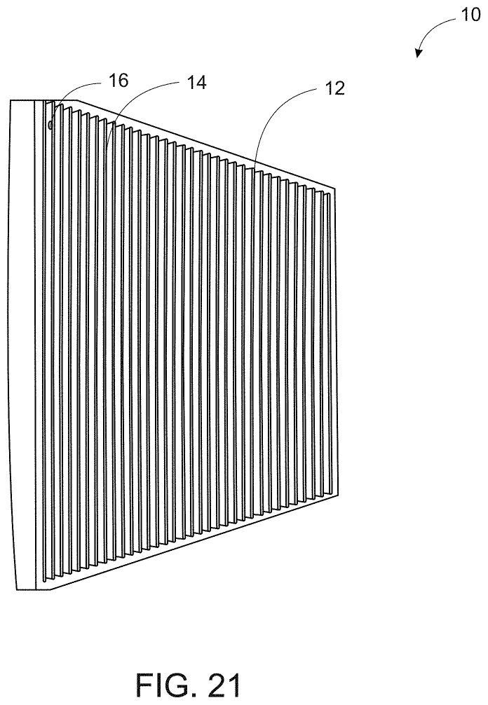

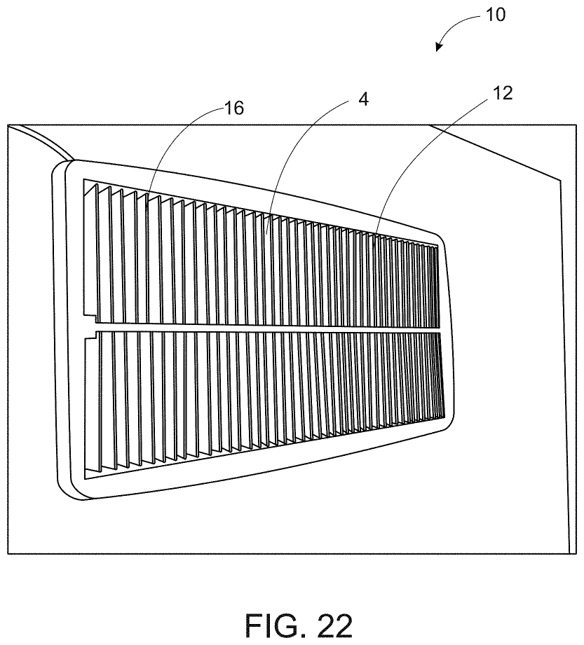

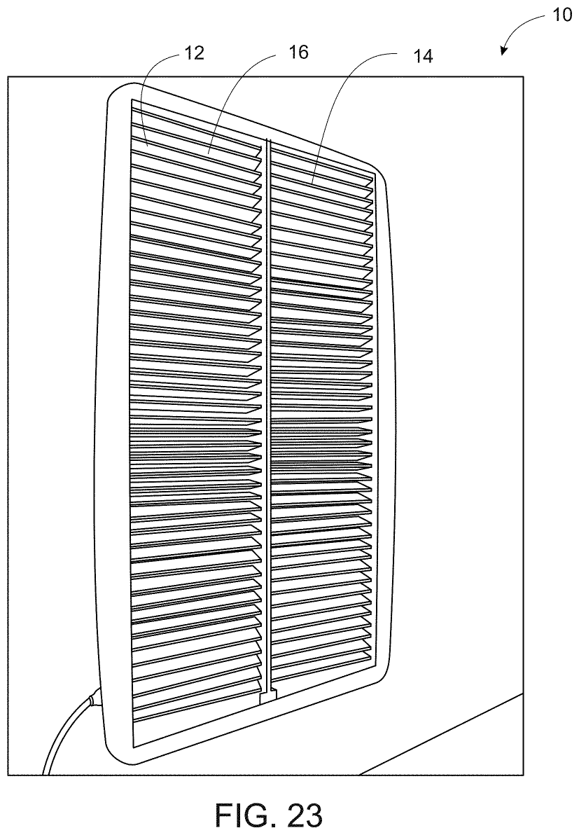

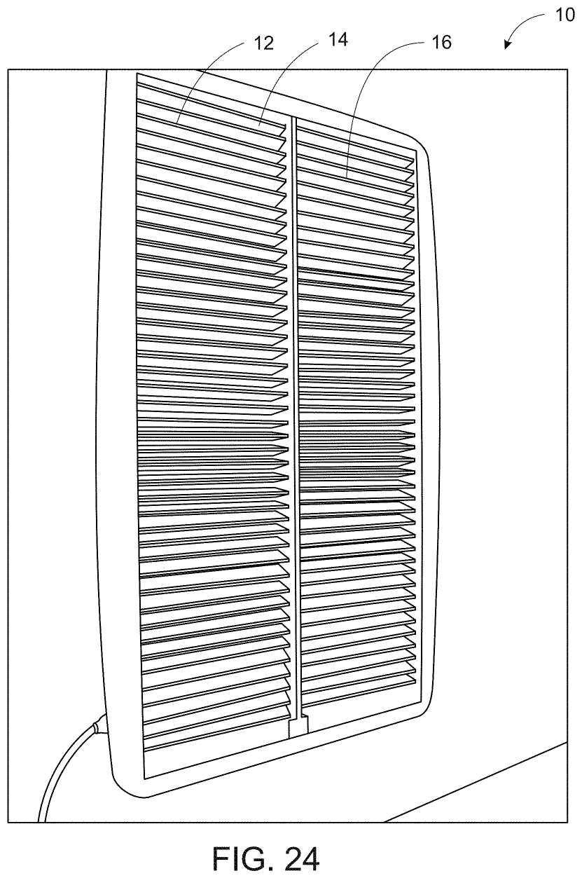

FIGS. 20 through 24 show additional novel cabin air filters 10 incorporating the matrix membrane 12, the flexible matrix 14, and the ultraviolet radiation or lights 16 attached to the matrix 14. Again, a string or one-dimensional strand of UV lights 16 can be incorporated into the novel cabin air filter 20 with or without the matrix membrane 12. As with the examples above, the filtration system 100 will force the circulating air 28 across and through the novel cabin air filter 10 thereby cleaning and sterilizing the circulating air 28 from pollutants 24 and toxins 30.

Because the matrix 14 of the matrix membrane 12 is flexible, there are many other uses for the present disclosure besides incorporation into a standard cabin air filter 20. By sewing in or weaving the matrix membrane 12 into other products, the present disclosure can likewise be used to sanitize, clean and disinfect many other products, even if the air is not circulating through the product. For example, if the matrix membrane 12 is sewn or weaved into a blanket, and the blanket is laid out on the sheets of a bed, the ultraviolet lights and radiation 16 can sanitize the bed sheets. This type of use can be utilized in a hotel or hospital environment.

Additionally, the matrix membrane 12 with the incorporated ultraviolet lights 16 can be sewn into the drapes or other window coverings such that the air in the room can be sanitized by energizing the matrix membrane 12 and the ultraviolet lights. Further, because the matrix membrane 12 is flexible, it can be shaped to fit onto benches or seats (or other areas) of mass transit, airplanes, taxis, Uber or Lyft automobiles, public transportation, etc. where it can sanitize the surface of the fabric, and anything that comes in contact with the surface, in which it is installed.

The present disclosure matrix membrane 12 and/or UV lights 16 can be sewn or woven into a couch, chair, throws, tapestries, rugs, or other flooring, such that the ultraviolet radiation will work to sanitize the nearby area, by sanitizing the actual item that the flexible matrix membrane 12 has been placed no or over, or sewn or woven into, or sanitize the air within the vicinity. Other uses can be incorporated based on the flexibility (or non-flexibility) of the matrix membrane 12.

It will be understood that the embodiments of the present disclosure, which have been described, are illustrative of some of the applications of the principles of the present disclosure. Although numerous embodiments of this disclosure have been described above with a certain degree of particularity, those skilled in the art could alter the disclosed embodiments without departing from the spirit or scope of this disclosure.

All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosed system and methods.

Additionally, joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosed apparatus, system and methods as disclosed herein.

What is claimed is:

1. A cabin air filtration system intended to be coupled to a cabin of a vehicle, utilizing at least one radiation source to sterilize a toxin in the cabin air filtration system, thereby providing a sterilized air distribution environment throughout the cabin of said vehicle, comprising:
a cabin air filter, said cabin air filter sized and configured to be securely installed into a proper location in said vehicle;
a matrix membrane, said matrix membrane configured to be attached to said cabin air filter; and
a matrix, said matrix configured to secure said at least one radiation source, said at least one radiation source emitting radiation to sterilize said toxin;
wherein, once the cabin air filter containing said matrix membrane is securely installed in the vehicle and said cabin air filtration system forces said cabin air through said cabin air filter, said at least one radiation source on said matrix membrane will sterilize said toxin as said cabin air passes through the cabin air filter.

2. The cabin air filtration system of claim 1, wherein said cabin air filter is retrofitted from a standard cabin air filter.

3. The cabin air filtration system of claim 1, wherein said at least one radiation source is an ultraviolet light.

4. The cabin air filtration system of claim 3, wherein said ultraviolet light is in one of a UV-C, UV-B or UV-A wavelengths.

5. (canceled)

6. The cabin air filtration system of claim 4, wherein said ultraviolet light is in the 100 to 280 nm wavelength range.

7. The cabin air filtration system of claim 6, wherein said ultraviolet light is in the 250 to 262 nm wavelength range.

8. The cabin air filtration system of claim 1, wherein said toxin is at least one of a virus, a bacteria, a mold, a fungi, a rickettsia and/or a protozoa.

9. The cabin air filtration system of claim 1, wherein said matrix membrane is attached into said cabin air filter by sewing or weaving the matrix membrane into said cabin air filter, or otherwise attaching the matrix membrane to the cabin air filter.

10. The cabin air filtration system of claim 1, wherein said matrix membrane and said matrix are flexible.

11. The cabin air filtration system of claim 1, wherein said vehicle comprises one of an automobile, a marine vehicle or an airplane.

12. The cabin air filtration system of claim 1, wherein said proper location in said vehicle comprises behind or near a glove box or glove compartment.

13. A cabin air filter to be installed in a vehicle, utilizing at least one radiation source to sterilize a toxin in a cabin air filtration system, thereby providing a sterilized air distribution environment throughout the cabin of said vehicle, comprising:
a cabin air filter, said cabin air filter sized and configured to be securely installed into a proper location in said vehicle;
a matrix membrane, said matrix membrane configured to be attached to said cabin air filter; and
a matrix, said matrix configured to secure said at least one radiation source, said at least one radiation source emitting radiation to sterilize said toxin;
wherein, once the cabin air filter containing said matrix membrane is securely installed in the vehicle and said cabin air filtration system forces said cabin air through said cabin air filter, said at least one radiation source on said matrix membrane will sterilize said toxin as said cabin air passes through the cabin air filter.

14. The cabin air filter of claim 13, wherein said cabin air filter is retrofitted from a standard cabin air filter.

15. The cabin air filter of claim 13, wherein said at least one radiation source is an ultraviolet light.

16. The cabin air filter of claim 15, wherein said ultraviolet light is in one of a UV-C, UV-B or UV-A wavelengths.

17. (canceled)

18. The cabin air filter of claim 16, wherein said ultraviolet light is in the 100 to 280 nm wavelength range.

19. The cabin air filter of claim 18, wherein said ultraviolet light is in the 250 to 262 nm wavelength range.

20. (canceled)

21. The cabin air filter of claim 13, wherein said matrix membrane is attached into said cabin air filter by sewing or weaving the matrix membrane into said cabin air filter, or otherwise attaching the matrix membrane to the cabin air filter.

22. The cabin air filter of claim 13, wherein said matrix membrane and said matrix are flexible.

23. The cabin air filter of claim 13, wherein said vehicle comprises one of an automobile, a marine vehicle or an airplane.

24. (canceled)
